(19) United States
(12) Patent Application Publication
(10) Pub. No.: US 2021/0228454 A1
(43) Pub. Date: Jul. 29, 2021

(54) METHOD FOR PRODUCING COATED INORGANIC POWDERS

(71) Applicant: ISHIHARA SANGYO KAISHA, LTD., Osaka (JP)

(72) Inventors: Takeshi FUJIMURA, Osaka (JP); Shogo ISEYA, Osaka (JP)

(21) Appl. No.: 17/128,269

(22) Filed: Dec. 21, 2020

(30) Foreign Application Priority Data
Jan. 28, 2020 (JP) 2020-011457

(51) Int. Cl. A61K 8/02; B05D 1/18; B05D 7/24; A61K 8/29; A61K 8/34; A61Q 1/02; A61Q 1/12; A61Q 1/10; A61Q 1/08; A61Q 17/04; A61Q 19/00

(52) U.S. Cl. CPC A61K 8/022; B05D 1/18; B05D 7/24; A61K 8/29; A61K 8/345; A61K 2800/805; A61Q 1/12; A61Q 1/10; A61Q 1/08; A61Q 17/04; A61Q 19/00; A61Q 1/02

(57) ABSTRACT

A method for producing a coated inorganic powder by stirring and mixing an inorganic powder and an organic compound, thereby coating the particle surface of the inorganic powder with the organic compound, the method including making the coating amount of the organic compound relative to the inorganic powder uniform while reducing the inorganic powder and the organic compound which stick to the inner wall of a vessel is provided.

When stirring the inorganic powder and the organic compound in a vessel that is equipped with a stirring blade and has an opening portion, at least a first stirring step and a second stirring step after the first stirring are provided. When the speed of the stirring blade of the first stirring step is defined as V₁ (m/s) and the speed of the stirring blade of the second stirring step is defined as V₂ (m/s), the relationship of “V₁>V₂” is established.

### TECHNICAL FIELD

The present invention relates to a method for producing an inorganic powder coated with an organic compound.

### BACKGROUND ART

Various inorganic powders such as a titanium dioxide powder and a zinc dioxide powder are blended in makeup cosmetics such as foundations, eyeshadows, and cheek rouges, sunscreen cosmetics, and milky lotions. As such an inorganic powder, an inorganic powder whose surface is coated with various organic compounds is used for the purpose of improving adhesion to the skin and tactile feeling of powder.

For example, coated inorganic powders, which are subjected to, for example, silicone treatment (see “Patent Literature 1”), hydrophobic treatment such as metal soap treatment (see “Patent Literature 2”), and hydrophilic treatment such as amino acid treatment (see “Patent Literature 3”), have been known.

Also, as a coated inorganic powder that can be evenly applied to the skin and exhibits less dullness or color unevenness in addition to the provision of a skin care effect to powder, an inorganic powder coated with polyhydric alcohol such as glycerin has been proposed (see “Patent Literature 4”).

### CITATION LIST

### Patent Literatures

Patent Literature 1: JP S60-163973 A
Patent Literature 2: JP 2004-051550 A
Patent Literature 3: JP H08-48612 A
Patent Literature 4: JP2017-200887 A

### SUMMARY OF INVENTION

### Technical Problem

To coat (surface-treat) an inorganic powder with an organic compound, usually, an inorganic powder and an organic compound are put into a vessel, and then mixed and stirred. However, the inorganic powder and the organic compound cannot be evenly mixed, and a phenomenon occurs such as the inorganic powder and the organic compound which stick to the inner wall of the vessel. The phenomenon causes problems such as low recovery yield of the coated inorganic powder and nonuniform coating amount of the organic compound. Further, such a phenomenon also causes a problem that variation in the coating amount of the organic compound for each operation is large, thereby resulting in unstable operation.

The present invention has been achieved in consideration of the above problems, and it is an object of the present invention to provide the reduction of the nonuniformity of the coating amount of the organic compound relative to the inorganic powder while reducing the inorganic powder and the organic compound which stick to the inner wall of a vessel.

### Solution to Problem

As a result of intensive studies on the above problems, the present inventors have found that the above problems can be solved by controlling the stirring speed of a stirring blade when raw materials (an inorganic powder and an organic compound) are stirred and mixed in a vessel equipped with the stirring blade.

That is, the present invention is as follows.

[1] A method for producing a coated inorganic powder whose surface is coated with an organic compound, comprising stirring an inorganic powder and the organic compound in a vessel that is equipped with a stirring blade and has an opening portion,

wherein the stirring has at least a first stirring step and a second stirring step performed after the first stirring step; and

when a speed of the stirring blade in the first stirring step is defined as V₁ (m/s) and a speed of the stirring blade in the second stirring step is defined as V₂ (m/s), V₁ is larger than V₂.

[2] The method according to [1], wherein when a stirring time in the first stirring step is defined as t₁ (min) and a stirring time in the second stirring step is defined as t₂ (min), t₁ is smaller than t₂.
[3] The method according to [1] or [2], wherein an air or a gas is discharged from an inside of the vessel while an air or a gas is supplied from an outside of the vessel via the opening portion.
[4] The method according to any one of [1] to [3], wherein the organic compound is liquid or in liquid state.
[5] The method according to any one of [1] to [4], wherein a temperature of the inorganic powder and a temperature of the organic compound in the vessel during the stirring are less than 150° C.
[6] The method according to any one of [1] to [5], wherein the organic compound is a polyhydric alcohol and/or a polysaccharide.
[7] The method according to any one of [1] to [6], wherein the organic compound is glycerin.
[8] The method according to [7], wherein the glycerin is preheated from 30° C. to 100° C. and then stirred in the vessel.
[9] The method according to any one of [1] to [8], wherein the inorganic powder is a titanium dioxide powder.

### Advantageous Effects of Invention

According to the method of the present invention, the stable operation can be provided by reducing the nonuniformity of the coating amount of the organic compound while reducing an inorganic powder and the organic compound which stick to the inner wall of a vessel to avoid the reduction of a recovery yield.

### BRIEF DESCRIPTION OF DRAWINGS

FIG. 1 is a vertical cross-sectional view schematically illustrating a stirrer according to an embodiment of the present invention.

FIG. 2 is a top view schematically illustrating a stirring blade according to an embodiment of the present invention.

### DESCRIPTION OF EMBODIMENTS

In the present invention, an inorganic powder and an organic compound are stirred by a vessel that is equipped with a stirring blade and has an opening portion, thereby coating the particle surface of the inorganic powder with the organic compound. Herein, the “coating” may be such that the entire inorganic powder is coated with a layer of the organic compound as a coating film or may be a state in which a part of the coating film has a hole. Also, the coating film may be, for example, a discontinuous coating film which is present in the shape of island. The shape of the coating film is not particularly limited.

The inorganic powder refers to a powder which is chemically inorganic. The inorganic powder may be natural mineral, or an inorganic powder artificially synthesized by using a compound of zinc, titanium, lead, iron, copper, chromium, or the like as a raw material. Examples of the inorganic powder include white pigments (e.g., zinc flower (zinc oxide), titanium dioxide, and white lead), red pigments (e.g., red iron oxide, vermilion, and cadmium red), yellow pigments (e.g., chrome yellow, ocher, cadmium yellow, and titanium yellow), green pigments (e.g., emerald green, and chromium oxide green), blue pigments (e.g., Prussian blue, and cobalt blue), violet pigments (e.g., manganese violet, and mars violet), black pigments (e.g., titanium black, and iron black), and transparent white pigments (which are also referred to as “extender pigment”, e.g., silica white, alumina white, white clay, and calcium carbonate), but are not limited to these pigments. The inorganic powder includes transparent pigments such as titanium oxide fine particles, and tin oxide fine particles. The inorganic powder further includes pigments of metals such as aluminum, gold, silver, and copper.

The particle size of the inorganic powder is not particularly limited. For example, an inorganic powder having an average primary particle size from about 0.01 μm or more to 1.0 μm or less can be used.

The average primary particle size of the inorganic powder can be measured by an electron microscope method. Specifically, an image of the inorganic powder is taken by means of a transmission electron microscope (H-7000, manufactured by Hitachi, Ltd.), and image processing is performed by means of an automatic image analyzer (LUZEX AP (registered trademark), manufactured by NIRECO). Then, the primary particle sizes are measured for 2,000 particles, and the average value thereof is defined as the average primary particle size of the inorganic powder.

The shape of the inorganic powder is not particularly limited, and an inorganic powder having any shape, for example, a spherical-shaped powder, a rod-shaped powder, an acicular-shaped powder, a spindle-shaped powder, and a plate-shaped powder can be used. As for the above average primary particle size of an inorganic powder having a shape other than the spherical shape, in the case of the rod-shaped powder, acicular-shaped powder, and spindle-shaped powder, the average primary particle size is defined by the average of lengths of the short axis side, whereas in the case of the plate-shaped powder, the average primary particle size is defined by the average of lengths of the diagonal line on the surface.

In a case of using a titanium dioxide powder for a pigment as the inorganic powder, the average primary particle size of the titanium dioxide powder is preferably from 0.15 μm or more to 0.6 μm or less, and more preferably from 0.2 μm or more to 0.4 μm or less in terms of hiding power and the like. The crystal structure thereof is not particularly limited, and an anatase-type, a rutile-type, a brookite-type, or the like can be used. In terms of inhibiting decomposition of the organic compound which is a coating substance, a rutile-type is preferable.

As such a titanium dioxide powder, those produced by any methods of, what is called, a sulfate process and a chloride process can be employed. The titanium dioxide powder whose particle surface has been coated with various inorganic compounds may also be used. Examples of the inorganic compound include metal oxides and/or metal hydrous oxides of silicon, aluminum, titanium, zirconium, tin, and antimony.

The above organic compound is not particularly limited as long as it can be used for the surface treatment of the inorganic powder. In terms of ease of coating and sticking to the inner wall of a vessel, an organic compound that is liquid at room temperature, or an organic compound that comes to be liquid by heating at the time of use is preferably used. The organic compound that comes to be in liquid state by emulsifying the organic compound or dissolving the organic compound in a solvent may be used. Examples of the organic compound include organic compounds such as polyhydric alcohol-based organic compounds, amine-based organic compounds, silicone-based organic compounds, metal soaps, carboxylic acid-based organic compounds, and polysaccharides (specifically, trimethylolmethane, trimethylolethane, trimethylolpropane, pentaerythritol, dimethylethanolamine, triethanolamine, dimethyl silicone, methyl hydrogen silicone, zinc octoate, stearic acid, oleic acid, glycerin, and the like). In the present invention, polyhydric alcohols and/or polysaccharides are preferable. As the polyhydric alcohol, at least one selected from glycerin, diglycerin, 1,3-butylene glycol, dipropylene glycol, pentylene glycol, and hexylene glycol is preferably used. As the polysaccharide, at least one selected from xanthan gum, tamarind seed gum, guar gum, gum arabic, dextran, dextrin, pullulan, and cyclodextrin is preferably used.

An organic compound, which has been heated in advance (i.e., preheated), may also be put into the vessel, and stirred. In a case of using glycerin as the organic compound, the heating (i.e., preheating) temperature of glycerin is preferably from 30° C. to 100° C., and more preferably from 40° C. to 60° C. Such heating reduces the viscosity of glycerin, thereby making the coating amount of glycerin more uniform.

The grade of glycerin is not particularly limited. Examples thereof include purified glycerin, food additive glycerin, concentrated glycerin (under the condition of Japanese Pharmacopoeia), glycerin (under the condition of Japanese Pharmacopoeia), concentrated glycerin for cosmetics, and glycerin for cosmetics.

A known stirrer (for example, a stirrer equipped with a stirring blade) can be used for stirring. An appropriate apparatus can be selected depending on the treatment amount, viscosity, and the like. A known stirring blade can be used for the stirring blade. For example, a stirring blade of a paddle type stirring blade, a propeller type stirring blade, an anchor type stirring blade, or the like can be used. These stirring blades may be used alone, or used in combination. Use of two or more types of stirring blades, for example, use of an upper blade and a lower blade allows mixing in a short time, and thus is more preferable. Further, commercially available blades, for example, an ST blade, a Y1 blade, a Z0 blade, an SR blade, a CK blade, a PO blade, a VL/SR blade, a PE blade, a A0 blade, an S0 blade, and a B0 blade, all of which are manufactured by Nippon Coke & Engineering Co., Ltd. can be used.

In the present invention, stirring is performed by dividing it in at least two stages. That is, the inorganic powder and the organic compound are stirred in the first stirring (the first stirring step), and subsequent stirring is performed (the second stirring step). After the second stirring step, stirring can be supplementary performed (for example, the third stirring step, and the fourth stirring step). Also, it is preferable to perform a subsequent second stirring step and the like by using a vessel used in the first stirring step, but the vessel may also be changed.

As for the above stirring, at least the first stirring step and the subsequent second stirring step are provided. When the speed of the stirring blade in the first stirring step is defined as V₁ (meter/sec (m/s)), and the speed of the stirring blade in the second stirring step is defined as V₂ (meter/sec (m/s)), V₁ is larger than V₂ (i.e., “V₁>V₂”). The speed of the stirring herein refers to the circumferential speed of the stirring blade. More preferably, “V₁≥V₂×1.5”, that is, V₁ is 1.5 times or more V₂, and even more preferably, V₁≥V₂×2.0, that is, V₁ is 2.0 times or more V₂.

Stirring of the inorganic powder and the organic compound in the first stirring step can be performed in any order. That is, the addition of the organic compound can be performed while the inorganic powder is stirred, or the addition of the inorganic powder can be performed while the organic compound is stirred. Also, the inorganic powder and the organic compound may be added and stirred at the same time. In particular, in terms of operability, it is preferred to add the organic compound while the inorganic powder is stirred. The amount of organic compound added is usually preferably about 0.1 to 10 mass %, more preferably 0.1 to 5 mass %, and even more preferably 0.3 to 3 mass % relative to the amount of the inorganic powder.

The first stirring is preferably performed at a high speed. Specifically, the first stirring is preferably performed at a circumferential speed of 30 to 50 m/s, and more preferably at a circumferential speed of 35 to 45 m/s. On the other hand, the second stirring is preferably performed at a speed slower than the speed of the first stirring, specifically, at a circumferential speed of 10 to 30 m/s, and more preferably at a circumferential speed of 15 to 25 m/s. The circumferential speed herein strictly refers to the circumferential speed when a stirring blade combining the ST blade and SR blade manufactured by Nippon Coke & Engineering. Co., Ltd. is used.

As for the stirring time of the first stirring step and the second stirring step (time required for stirring both the inorganic powder and the organic compound), when the stirring time of the first stirring step is defined as t₁ (min), and the stirring time of the second stirring step is defined as t₂ (min), “t₁<t₂” is preferable. More preferably, “t₁×1.5≤t₂”, that is, t₂ is 1.5 times or more t₁, and even more preferably, “t₁×2.0≤t₂”, that is, t₂ is 2.0 times or more t₁. For example, the time of the first stirring step is preferably 2 to 5 minutes, whereas the time of the second stirring step is preferably 5 to 15 minutes.

The reason for stirring in two stages in this way is as follows. First, stirring at a relatively rapid speed in the initial stage of stirring in the stirring of the organic compound and the inorganic powder allows the organic compound to be evenly dispersed in the entire vessel at a certain level. When the state of stirring becomes this state, stirring is then performed at a low speed, and thus the surface treatment of the inorganic powder with the organic compound is completed. Such an operation can prevent uneven distribution of the organic compound in the vessel, and thus can ensure the uniformity of the coating amount of the organic compound relative to the inorganic powder in the same vessel. Further, performing minimal high-speed stirring reduces an increase in the temperature caused by friction heat generated between inorganic powders, and thus reduces the amount of water evaporated derived from raw materials (the organic compound and the inorganic powder). Also, this can also reduce the thermal denaturation of the organic compound.

Further, in the present invention, an opening portion provided in the vessel allows water vapor generated in the interior of the vessel to be discharged from the opening portion to the outside of the vessel as described above. This reduces condensation of water vapor on the inner wall of the vessel, and thus can reduce the inorganic powder and the organic compound which stick to the inner wall of the vessel. The opening portion is preferably provided at two or more positions of the vessel. The size of the opening portion can be appropriately set. At this time, it is more preferable that while air or gas is supplied (introducing air or gas such as oxygen or nitrogen from the outside of the vessel) from the outside of the vessel via the opening portion, air or gas is discharged from the inside of the vessel via another opening portion. Further, a known suction-discharge device may be attached to the opening portion. Examples of the suction-discharge device include a vacuum pump. Promoting discharge of water vapor in the vessel in this way can further reduce the inorganic powder and the organic compound which stick to the inner wall of the vessel.

The problems such as the inorganic powder and the organic compound which stick to the inner wall of the vessel, nonuniform coating amount of the organic compound relative to the inorganic powder, and the thermal denaturation of the organic compound are particularly significant in a case of using glycerin as the organic compound. Accordingly, the present invention is particularly suitable for, particularly, a case of coating an inorganic powder with glycerin. In a case of using glycerin, the temperature (specifically, material temperature) of the inorganic powder and glycerin during the stirring are preferably less than 150° C. Such a temperature can reduce the denaturation of glycerin, and also can treat the inorganic powder with glycerin more uniformly.

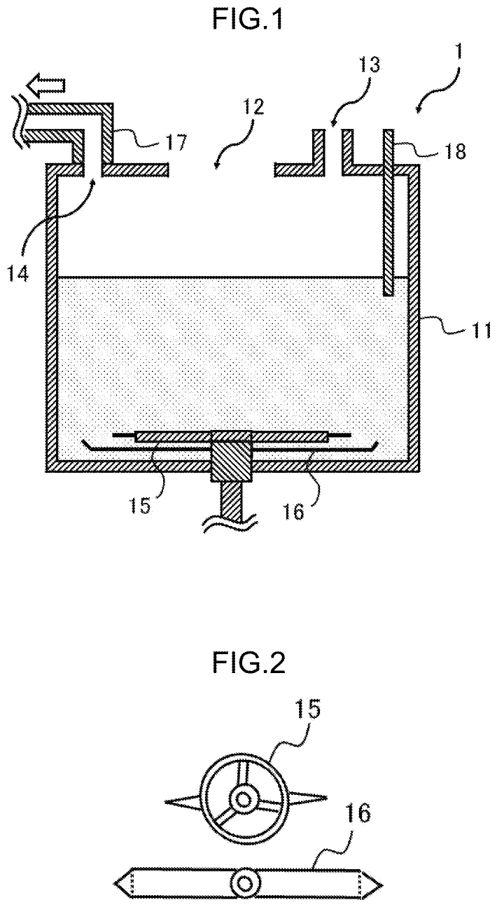

Even in a case of using an organic compound other than glycerin, by setting the temperature (specifically, material temperature) of the inorganic powder and the organic compound during stirring to less than 150° C., the denaturation of the organic compound can be reduced, which is preferable. The temperature (specifically, material temperature) of the inorganic powder and the organic compound during stirring can be measured by a thermocouple provided in a vessel of a stirring mixer. FIG. 1 illustrates a state in which a thermocouple 18 is inserted in contents (specifically, inorganic powder and organic compound) contained in a vessel 11 in a Henschel mixer 1 which is one aspect of the stirring mixer of the present invention.

After the inorganic powder and the organic compound are stirred by a vessel equipped with a stirring blade to coat the particle surface of the inorganic powder with the organic compound in this way, the coated inorganic powder may be taken out and dried as necessary. The drying temperature is preferably about 80° C. to 150° C. The particle size may also be appropriately adjusted by means of a known grinder, classifier, or the like.

In a case of using a titanium dioxide powder as the inorganic powder, and glycerin as the organic compound, a coated inorganic powder having “L value” of powder in the Hunter Lab color space (Lab colorimetric system) of 98.0 to 99.5, “a value” of −0.5 to 0.0, and “b value” of 1.0 to 1.5 can be produced. A coated inorganic powder having the “L value”, “a value”, and “b value”, described above can be suitably used for cosmetics and the like. The above “L value”, “a value”, and “b value” can be measured by means of a colorimetric color difference meter or the like. For example, a spectroscopic colorimeter SD5000, manufactured by Nippon Denshoku Industries Co., Ltd. can be used.

Additionally, in a case of using a titanium dioxide powder as the inorganic powder, and glycerin as the organic compound, a coated inorganic powder having a pH of 6.5 to 8.0 can be produced. The coated inorganic powder having the pH described above can be suitably used for cosmetics and the like. The pH herein refers to the pH of a dispersion when the coated inorganic powder is dispersed in water. For example, the pH can be measured by means of a pH meter D-71, manufactured by HORIBA, Ltd., or the like.

### EXAMPLES

The present invention will be described in detail with reference to the examples and the comparative examples. However, the present invention is not limited to these examples.

### Example 1

150 kg of a titanium dioxide powder (CR-50, manufactured by Ishihara Sangyo Kaisha, Ltd.) was put into a Henschel mixer (manufactured by Nippon Coke & Engineering. Co., Ltd., stirring blade: SR blade and ST blade) and stirred at a circumferential speed of 40 m/s. FIG. 1 illustrates a vertical cross-sectional view of a Henschel mixer 1 which is a stirring mixer used in “Example 1”. Two opening portions (i.e., an opening portion 13 and an opening portion 14) are provided in the upper part of the vessel 11 of the Henschel mixer 1. The opening portion 14 is connected to a sucking pump (not illustrated) by a hose 17. During stirring, operation of the sucking pump introduces the outside air from the outside of the vessel 11 via the opening portion 13, and air or water vapor and the like in the vessel are discharged to the outside of the vessel from the opening portion 14 via the hose 17. Two types of stirring blades (a SR blade 16 and a ST blade 15) are provided on the same axis in the bottom part of the vessel 11 of the Henschel mixer 1. FIG. 2 illustrates a top view of the ST blade 16 and the ST blade 15 of the present example.

1.05 kg of concentrated glycerin for cosmetics (manufactured by Sakamoto Yakuhin kogyo Co., Ltd.), which has been preheated to 40° C., was added to the titanium dioxide powder from an input port 12 over 30 seconds without dilution, and further stirred for 3 minutes (i.e., first stirring step). Next, the circumferential speed was changed to 20 m/s in the same vessel, and further stirred for 5 minutes (i.e., second stirring step), thereby obtaining “Sample A”. The material temperature during the stirring step was kept to less than 150° C.

### Comparative Example 1

In “Example 1” above, the pattern of stirring was changed to a pattern of stirring at a low speed in the initial stage, and then switching to stirring at a high speed in the middle of stirring. Specifically, the circumferential speed and the time were changed to 20 m/s and 5 minutes in the first stirring step, and the circumferential speed and the time were changed to 40 m/s and 3 minutes in the second stirring step. By taking the same procedures as in “Example 1” except for the above conditions, “Sample B” was obtained.

### Comparative Example 2

In “Example 1” above, stirring was performed in a non-open state (a state in which the opening portion of the Henschel mixer is closed). Further, the circumferential speed and the time of the first stirring step were set to 40 m/s and 7 minutes, and the circumferential speed and the time of the second stirring step were set to 20 m/s and 15 minutes. By taking the same procedures as in “Example 1” except for the above conditions, “Sample C” was obtained.

### Comparative Example 3

In “Example 1” above, the pattern of stirring was changed to a pattern of stirring at a low speed in the entire stirring, and the circumferential speed of stirring was set to 20 m/s, and the time of stirring was set to 15 minutes. Further, stirring was performed in a non-open state. By taking the same procedures as in “Example 1” except for the above conditions, “Sample D” was obtained.

<Measurement Method of “C Amount”>

The “C amount” was measured by means of an automatic elemental analyzer (Vario ELIII, manufactured by Elementar, combustion tube temperature: 950° C., reduction tube temperature: 600° C.).

<Calculation of Standard Deviation>

The uniformity of the coating amount of the organic compound relative to the inorganic powder in the same vessel is evaluated as follows. First, the six samples are collected at a plurality of different positions in a vessel for an inorganic powder which has been surface-treated with an organic compound in the same vessel. The “C (carbon) amount” was measured for each sample by means of an elemental analyzer, and the standard deviation of the coating amount of the organic compound was calculated from the measurement results.

<Ratio of Coated Inorganic Powder Sticking to Vessel in Each Sample>

The amount sticking to the vessel (i.e., difference between the total amount of raw materials (i.e., inorganic powder and organic compound) which were put into the vessel and the amount of the obtained sample) was calculated. The ratio of the coated inorganic powder sticking to a vessel was calculated by dividing the amount sticking to the vessel by the total amount of raw materials which were put into the vessel.

Table 1 shows each production condition of the “Samples A to D”, and the standard deviation of the treatment amount of the organic compound in each sample. As for the coated inorganic powder sticking to the inner wall in each sample of Table 1, the case where its ratio was less than 10 mass % was evaluated as “∘”, and the case where its ratio was 10 mass % or more was evaluated as “x”.

TABLE 1

| | | | | | | | Evaluation | |
|---|---|---|---|---|---|---|---|---|
| | | | | | | | of ratio of | |
| | | Production condition | | | | | coated | Standard |
| | | | First stirring step | | Second stirring step | | inorganic | deviation |
| | | | circumferential | | circumferential | | powder | of |
| | | Open | speed | Time | speed | Time | sticking to | coating |
| | Sample | state | (m/s) | (min) | (m/s) | (min) | inner wall | amount |
| Example 1 | A | Yes | 40 | 3 | 20 | 5 | ∘ | 0.014 |
| Comparative | B | Yes | 20 | 5 | 40 | 3 | ∘ | 0.046 |
| Example 1 | | | | | | | | |
| Comparative | C | No | 40 | 7 | 20 | 15 | x | — |
| Example 2 | | | | | | | | |
| Comparative | D | No | 20 | 15 | — | — | ∘ | 0.195 |
| Example 3 | | | | | | | | |

<Regarding Ratio of Coated Inorganic Powder Sticking to Vessel>

In “Example 1”, the ratio of the coated inorganic powder sticking to the vessel can be reduced. This also indicates that the ratio of raw materials (i.e., inorganic powder and organic compound which were put into the vessel as raw materials) sticking to the vessel can be reduced. The ratio of the coated inorganic powder sticking to the vessel could be reduced to a preferable range of less than 10 mass %, specifically, a range of 1 mass % to 3 mass %. On the other hand, in “Comparative Example 2” which performs stirring in a closed state at a relatively high speed as in the conventional method, the ratio of the coated inorganic powder sticking to the vessel was a range of 10 mass % to 20 mass %.

<Regarding Open State>

As shown in Table 1 above, comparing “Example 1” (i.e., “Sample A”) with “Comparative Example 2” (i.e., “Sample C”), it has been confirmed that “Example 1” which performed stirring in an open state can reduce the coated inorganic powder sticking to the inner wall of the vessel. On the other hand, it has been also confirmed that “Comparative Example 2” which performed stirring in a non-open state results in occurrence of the coated inorganic powder sticking to the inner wall of the vessel. As for “Comparative Example 3” (i.e., “Sample D”), although stirring was performed in a non-open state as in “Comparative Example 2”, the coated inorganic powder sticking to the inner wall has not been observed. This can be understood as the reduction of the water vapor from raw materials attributed to mildly stirring at a relatively slow circumferential speed.

<Regarding Stirring Condition>

As shown in Table 1, in “Example 1” (i.e., “Sample A”) which performed stirring in two stages of which an initial stage was performed by stirring at a high speed and its next stage was performed by being switched to stirring at a low speed in the middle of stirring at the high speed, it has been confirmed that the standard deviation of the coating amount of the organic compound is 0.03 or less, and the coating amount of the organic compound relative to the inorganic powder in the same vessel comes to be uniform.

On the other hand, as shown in Table 1, it has been also confirmed that, in “Comparative Example 1” (“Sample B”) which performed stirring at a low speed in the initial stage or in “Comparative Example 3” (i.e., “Sample D”) which performed stirring at a low speed in the entire stirring, the standard deviation (i.e., uniformity of the coating amount of the organic compound in the same vessel) is large. This can be understood as being caused by the reason that the evenness of mixing during stirring at a low speed in the initial stage is insufficient.

As for “Sample A”, its “L value”, “a value”, and “b value” of powder in the Lab colorimetric system and its pH were measured as described below.

<Measurement Method of “L Value”, “a Value”, and “b Value” of Powder in Lab Colorimetric System>

5 g of “Sample A” was filled in an aluminum ring (outer diameter: 38 mm, inner diameter: 34 mm, thickness: 5 mm), and press-molded by a compression molding machine at a pressure of 10 tf. Then, the measurement was performed by means of a spectroscopic colorimeter SD5000, manufactured by Nippon Denshoku Industries Co., Ltd.

<Measurement Method of pH>

To 50 g of a sample A, 250 ml of pure water was added, and the mixture was stirred by means of a fiber mixer MX-X700, manufactured by Panasonic Corporation for 3 minutes. After stirring, the pH of the obtained slurry was measured by means of a pH meter D-71, manufactured by HORIBA, Ltd.

The “L value”, “a value”, and “b value” of powder of “Sample A” in the Lab colorimetric system was 98.8, −0.4, and 1.4, respectively. In addition, the pH was 6.9. The coated inorganic powder having the “L value”, “a value”, and “b value”, and pH, as described above can be suitably used for cosmetics and the like.

### INDUSTRIAL APPLICABILITY

In the present invention, an inorganic powder can be uniformly coated with an organic compound, and such coated inorganic powder can be used for various applications such as cosmetics.

### REFERENCE SIGNS LIST

1 Henschel mixer
11 Vessel
12 Input port
13,14 Opening portion
15 ST blade
16 SR blade
17 Hose
18 Thermocouple

1. A method for producing a coated inorganic powder whose surface is coated with an organic compound, comprising stirring an inorganic powder and the organic compound in a vessel that is equipped with a stirring blade and has an opening portion,
wherein the stirring has at least a first stirring step and a second stirring step performed after the first stirring step; and
when a speed of the stirring blade in the first stirring step is defined as V₁ (m/s), and a speed of the stirring blade in the second stirring step is defined as V₂ (m/s), V₁ is larger than V₂.

2. The method according to claim 1, wherein when a stirring time in the first stirring step is defined as t₁ (min) and a stirring time in the second stirring step is defined as t₂ (min), t₁ is smaller than t₂.

3. The method according to claim 1, wherein an air or a gas is discharged from an inside of the vessel while an air or a gas is supplied from an outside of the vessel via the opening portion.

4. The method according to claim 1, wherein the organic compound is liquid or in liquid state.

5. The method according to claim 1, wherein a temperature of the inorganic powder and a temperature of the organic compound in the vessel during the stirring are less than 150° C.

6. The method according to claim 1, wherein the organic compound is a polyhydric alcohol and/or a polysaccharide.

7. The method according to claim 1, wherein the organic compound is glycerin.

8. The method according to claim 7, wherein the glycerin is preheated from 30° C. to 100° C. and then stirred in the vessel.

9. The method according to claim 1, wherein the inorganic powder is a titanium dioxide powder.

10. The method according to claim 2, wherein an air or a gas is discharged from an inside of the vessel while an air or a gas is supplied from an outside of the vessel via the opening portion.

11. The method according to claim 2, wherein the organic compound is liquid or in liquid state.

12. The method according to claim 2, wherein a temperature of the inorganic powder and a temperature of the organic compound in the vessel during the stirring are less than 150° C.

13. The method according to claim 2, wherein the organic compound is a polyhydric alcohol and/or a polysaccharide.

14. The method according to claim 3, wherein the organic compound is a polyhydric alcohol and/or a polysaccharide.

15. The method according to claim 4, wherein the organic compound is a polyhydric alcohol and/or a polysaccharide.

16. The method according to claim 5, wherein the organic compound is a polyhydric alcohol and/or a polysaccharide.

17. The method according to claim 2, wherein the organic compound is glycerin.

18. The method according to claim 2, wherein the inorganic powder is a titanium dioxide powder.

19. The method according to claim 3, wherein the inorganic powder is a titanium dioxide powder.

20. The method according to claim 4, wherein the inorganic powder is a titanium dioxide powder.
